(19) United States
(12) Patent Application Publication
(10) Pub. No.: US 2025/0228304 A1
(43) Pub. Date: Jul. 17, 2025

(54) STOCKING

(71) Applicant: INFINIRI AG, Luzern (CH)

(72) Inventors: Ian ECHLIN, Luzern (CH); Detlev DOBRINSKI BRINKER, Riehen (CH); Peter HENDE, Iserlohn (DE)

(21) Appl. No.: 18/844,591

(22) PCT Filed: Mar. 7, 2023
(86) PCT No.: PCT/EP2023/055692

§ 371 Date: Sep. 6, 2024

(30) Foreign Application Priority Data
Mar. 8, 2022 (WO) PCT/EP2022/055924

(51) Int. Cl. A41B 11/02

(52) U.S. Cl. CPC A41B 11/02; A41B 2400/32

(57) ABSTRACT

Stocking 10 with a foot part 11 for accommodating a human foot, wherein a support element 14 is positioned on a sole area 13 of the foot part such that the support element comes to lie under at least one metatarsal when the stocking is worn on the foot, wherein the support element has a curvature 15 facing the inside of the stocking.

The present invention relates to a stocking with a foot part for accommodating a human foot.

Hallux valgus or other foot malpositions often occur as a result of incorrect footwear. If the deformity is severe, it can only be corrected by surgical treatment. Measures to prevent and correct the deformity at an early stage are of great interest. Means for this, such as orthopaedic shoes, are often not elegant and each pair of shoes is relatively expensive. A flexibly applicable and inexpensive means is the gluing of elastic tapes to the skin of the foot. Wearing tapes can easily irritate the skin, so that this means is often unsuitable for long-term use. European patent specification EP 3429514 B1 discloses a medical stocking that can be used to treat foot deformities such as hammer toes. This medical stocking has at least two separate chambers for accommodating the toes, with a first chamber accommodating the big toe alone, and it has various elastic tension bands, one of which extends laterally on the foot from the big toe to the heel.

The object of the present invention was to provide an alternative solution for the treatment or prevention of foot deformities, or at least for the comfort of the foot. A more specific object of the present invention was to provide an easy-to-use means. In particular, the invention is intended to avoid disadvantages of known means for correcting a foot deformity.

In particular, the invention aims to attract consumers who are reluctant to use conventional orthopaedic products. The aim is to provide a means for correcting or preventing foot deformity that does not require professional help for application and preferably does not require any additional adhesives, tapes, etc. for correct application.

According to the invention, this object is solved by a stocking according to claim 1. The stocking according to the invention has a foot part for accommodating a human foot. In the context of the present invention, the term stocking should also be understood to mean a lifestyle or health stocking, a stocking of any length, or even a pair of tights, as long as the stocking can accommodate at least one foot in the foot part. The foot part comprises an area to accommodate the toes, an area to accommodate the midfoot and an area to accommodate the heel. Depending on the length, the stocking comprises no leg part or a leg part that extends above the ankles, below the knee, above the thigh, above the hip, etc. A support element is positioned on a sole area of the foot part in such a way that the support element comes to lie under at least one of the metatarsals when the stocking is worn on the foot. For example, the support element can be positioned under all five metatarsals. The support element can, for example, also come to lie under a single one of the metatarsals one to five or under a group of metatarsals. The support element has a curvature facing towards the inside of the stocking.

The inventors have recognized that the support element in a soft version already has the pleasant effect of preventing pressure points. It can provide a pleasant wearing sensation when walking and standing. The stocking according to the invention can easily be made so thin and flexible that it fits into a shoe.

The support element can be in one or more parts. A one-piece form of the support element can have the shape of an ellipse, an oval or a circle in plan, i.e. viewed perpendicular to the sole. Other rounded organic shapes or angular shapes such as a diamond shape are also possible. A multi-part form of the support element can, for example, have parts that support a single metatarsal.

The support element can be made of medical silicone, for example. This allows it to be worn directly on the skin. The support element can also comprise foam rubber, for example. The support element can have a textile cover. The support element can comprise textile or even consist entirely of textile material.

Exemplary embodiments are derived from the features of claims 2 to 15.

In one embodiment, the support element is designed and positioned such that the second to fourth metatarsals are raised relative to the first and fifth metatarsals when a wearer of the stocking strikes the ground.

By holding the support element underneath metatarsals 2 to 4 by the stocking, the position of the metatarsals is guided towards their natural shape of a transverse arch position. By wearing the stocking, the development of a malposition can be prevented or an existing slight malposition can be treated.

The support element can be designed in the form of a so-called pelot, for example. In a typical dimensioning of the support element for the foot of an adult person, for example, the height of the curvature can be 7 millimeters. A width of 3.5 centimetres and a length of 5 centimetres are suitable for this dimensioning.

In particular, the support element can be dimensioned in such a way that by wearing the stocking the metatarsals two to four (metatarsals 2, 3, 4) are elevated by 1 to 15 millimeters compared to their position without support from the support element. The amount by which an individual metatarsal is elevated can be different for each of the metatarsals 2, 3 and 4 and can be within the specified range. A personalized adaptation to a specific wearer of the stocking is possible here, for example raising the second metatarsal by 5 mm, the third metatarsal by 10 mm and the fourth metatarsal by 3 mm. The support element can be designed as a less compressible element. Alternatively, the support element can be designed as a thickened part of a padding which extends over a larger sole area and in which the thickened part comes to lie under the metatarsals two to four.

In one embodiment, the support element (14) is positioned behind the metatarsophalangeal joints when the stocking is worn on the foot.

This embodiment has the effect of achieving the natural arch shape of the foot in the longitudinal direction and in the cross-sectional direction. Furthermore, this embodiment prevents pressure points on the particularly sensitive areas under the metatarsophalangeal joints. In combination with a variant in which the support element is designed as a thickened part of a padding, the thickened part of the padding lies behind the metatarsophalangeal joints. The remaining padding can be positioned under the metatarsophalangeal joints and even extend forwards beyond the metatarsophalangeal joints.

In one embodiment, the support element comprises a first area with a first height and a second area with a second height. The second area is arranged in an island shape in the first area or the second area is arranged at an edge of the first area. The second height exceeds the first height by at least a factor of two.

The support element can, for example, be designed completely flat on its underside and have even transitions at the edge and between the two height areas on its upper side, i.e. on the side facing the foot. Overall, this creates an area with a height of “0” on the sole of the foot, where the foot rests on the fabric of the stocking without the support element in between. This creates an area with a height of “1”, in which a minimal support effect and lifting of the footbed is achieved. The area with height “1” also has a cushioning effect, so that pressure points in this area can be avoided. This also creates a central area with a height of “2” in which the maximum support effect of the support element occurs. The outer contour of the first area can, for example, be arranged in such a way that the toes come to lie outside the first area of the support element, for example by the front edge of the first area extending at an angle to the foot axis so that the first area of the support element extends further forward in the area of the big toe than in the area of the little toe.

In particular, this embodiment can be combined with a heel pad and a tension device, with features of the heel pad and tension device being discussed in more detail below.

In one embodiment, the support element consists of a material with a Shore hardness in the range 0 to 50. In particular, a Shore hardness close to 5, also in the range 4 to 8, is preferred.

As the inventors have recognized, the support element has good resilience in this hardness range, i.e. it retains a shape that maintains the support effect even after prolonged walking and standing. On the other hand, the support element is soft enough so that it is comfortable to wear on the foot and also provides cushioning and prevents pressure points. In particular, a support element made of a silicone rubber with a Shore hardness in the range of 4 to 10, especially a Shore hardness of 5, is effective as a support element and comfortable to wear on the foot. The support element can, for example, be made of a skin-compatible silicone with a Shore hardness in the range of 4 to 8.

In one embodiment of the stocking, a length of the sole area is defined from a first end for accommodating the toes to a second end for accommodating the heel. A width of the sole area is defined perpendicular to the length and parallel to the sole area. The support element is centered with respect to the width. In terms of length, the support element is positioned at a distance of ⅖ of the length from the first end and ⅗ of the length from the second end. This type of positioning makes it possible to provide the right stocking with a correctly positioned support element for different standard shoe sizes. Due to the length of the support element of, for example, approx. 5 cm, the support element protrudes approx. 2.5 cm beyond the stated position each at the front and back, but its center should be approximately at the stated position. Alternatively, the support element can also protrude further back, for example from the specified position 3.5 to 4.5 cm to the rear, resulting in a length of the support element of 6 to 7 cm. In this case, the highest point of the support element (instead of the center point) remains at the position measured ⅖ from the front, ⅗ from the rear. Due to this positioning of the support element on the sole area, the support element comes to lie in the desired position for the purposes of the present invention below the metatarsals and behind the metatarsophalangeal joints, i.e. slightly towards the heel when viewed from the toes.

In one embodiment of the stocking, the support element has a teardrop shape when viewed perpendicular to the sole area, wherein the teardrop shape is oriented such that a tapering area of the teardrop shape faces towards the heel when the stocking is worn on the foot. A raised area within this teardrop shape may, for example, have an oval shape or an organic shape, wherein a longitudinal extension of the oval or organic shape is oriented in a longitudinal direction of the foot.

In one embodiment, the stocking has a one-piece or multi-piece padding on the sole area.

Such padding can extend over parts of the sole of the foot or over the entire length and width of the sole of the foot.

Padding can extend over the area over which the metatarsals come to rest. In this case, the support element can be placed on or under the padding or integrated into the padding. In a multi-part version, for example, a separate pad can come to rest under the heel. Such a so-called heel pad cushions a point on the foot where the foot already has a natural cushion. Such a heel cushion can, for example, have a recess in a central area of the pad, for example so that the recess comes to rest below the heel bone. Padding can be positioned below the forefoot. Padding of the type discussed can be attached to the inside or outside of the stocking. Further variants of the padding are shown below in connection with the figures.

In one embodiment of the stocking with padding on the sole area, at least part of the padding is designed as forefoot padding. For example, the forefoot padding is arranged on the sole area in such a way that it comes to lie under the metatarsophalangeal joints when the stocking is worn on the foot.

This forefoot padding under the metatarsophalangeal joints 1-5 absorbs the impact and rolling motion. It absorbs pressure peaks from the metatarsophalangeal joints and cushions them.

In one embodiment of the stocking, the foot part of the stocking comprises a tensile element. The tensile element is arranged on the foot part or incorporated into the foot part such that the tensile element extends around the midfoot and exerts a contracting force on the midfoot when the stocking is worn on the foot. This contracting force on the midfoot is greater than the contracting force of the rest of the foot. Due to the use of an elastic material and a dimensioning slightly smaller than the foot, the remaining foot part, i.e. that part of the foot part which does not belong to the tensile element, can also be pulled slightly against the foot with a contracting force, which corresponds to a normal stretch effect of a stocking. The tensile element in the sense of the invention also acts with a higher tensile force acting locally in the area of the midfoot. This tensile force supports the effect of the support element to straighten the arch formed by the metatarsals.

The tensile element can, for example, be connected to the foot part over its surface, e.g. glued to the outside of the stocking. Alternatively, the tensile element, or at least parts of the tensile element, can be incorporated into the foot part, e.g. knitted or woven in. The tensile element can also be attached to the stocking in other ways, e.g. glued, welded, sewn on or riveted. The tensile element can also be inserted or attached in the stocking as an independent element, e.g. manufactured as a cast part.

For example, the tensile element can be limited to a zone approx. 4 cm wide, or 2 to 6 cm wide depending on the size of the foot, surrounding the midfoot. This leaves a zone in the toe area and in an area around the leg and heel area that is free of tensile elements.

In one embodiment of the stocking with a tensile element, the tensile element comprises band-shaped sections which converge in a cross shape in the sole area or which run parallel in the sole area or which converge in a cross shape in the area of the foot instep.

In one embodiment of the stocking, the tensile element comprises a band-shaped part which, when unwound, has the shape of the digit eight.

The cross-shaped area of the digit eight comes to rest under the sole of the foot. The loops of the eight wrap around the midfoot on both sides. The loops do not touch in the instep area. Elastic material in the area between the loops allows a tensile force to be developed. The band-shaped part in the form of the digit eight has the effect of a fixed brace, which reduces the angle of the metatarsophalangeal joint to the second toe ray and prevents the metatarsophalangeal joint from moving out of position.

The inventors have recognized that this shape interacts particularly effectively with the support element. In particular, the supporting muscles of the arch of the foot are strengthened to a high degree. The tension/pressure distribution on the foot is favorably distributed by the digit-of-eight sling. Surprisingly, this shape minimizes slippage of the tensile element relative to the foot by simple means. Furthermore, the inventors have recognized that this shape enables an aesthetically pleasing design of the stocking, especially if the tensile element stands out from the rest of the stocking in terms of colouring, light/dark difference or opacity/transparency.

In an alternative embodiment, the tensile element is topologically doubly connected, i.e. it is topologically equivalent to a circular ring. In this embodiment, in contrast to the tensile element discussed above, the tensile element does not have a cross-shaped converging point. The tensile element in this embodiment can be designed in such a way that, when the stocking is worn on the foot, the tensile element runs, so to speak, in two tracks under the sole of the foot transversely under the foot, wherein the two tracks are connected above the instep of the foot by a left-lying and a right-lying arc without the two arcs touching each other. In another version of a topologically double-connected tensile element, the tensile element forms a kind of tubular section that runs around the midfoot. When the stocking is worn on the foot, such a tensile element wraps around the foot in the area of the metatarsals and runs under the foot and is closed over the instep. This tensile element can also be approx. 2 to 6 cm wide, for example.

In one embodiment of the stocking with a tensile element, the tensile element comprises a first band-like section along a circumference extending around the foot part with less extensibility than the rest of the foot part. The tensile element further comprises a second elastic section, wherein the first and second sections together form a closed ring.

The contracting force comes from the elastic section, while the first section of the tensile element transmits this force and prevents the stocking from flexing at the points of the first section. This enables a localized force effect in the area of the metatarsals. The fact that the first section is band-like means that the force is applied over a large area and pressure points are avoided.

This embodiment can be combined in particular with the embodiment discussed above with a tensile element in the shape of the digit eight, wherein the first band-like section has the shape of the digit eight.

In one embodiment, the stocking comprises a clasp element which is designed to at least partially embrace the metatarsals and exert a force acting laterally on the foot when the stocking is worn on the foot.

Such a clasp element is an alternative solution to the tensile element. It exerts the force on the metatarsals through the inherent tension of the clasp element. In contrast to the tensile element, the clasp element can be open on one side, e.g. above the instep of the foot. The clasp element can be C-shaped, for example.

The invention further relates to a stocking according to claim 16.

A stocking of this type has a sole area which has a pad in the area of the forefoot, i.e. in the area of the so-called ball of the foot, within which the metatarsophalangeal joints of the toes are also located. The inventors have recognized that a forefoot pad alone in a stocking makes it very comfortable to wear. The stocking according to claim 16 can be combined with any of the features of claims 1 to 15. In particular, the padding in a stocking according to claim 16 can come to lie below the metatarsophalangeal joints when the stocking is worn on the foot.

The invention further relates to a manufacturing method according to claim 17 and a manufacturing method according to claim 18.

In a manufacturing method for manufacturing a stocking according to the invention, the manufacturing method comprises the steps of:
providing a stocking cover made of textile, wherein the stocking cover has a foot part with an inner surface,
providing a support element, and
fixing the support element to the inner surface of the foot part of the stocking cover.

The fixing step may involve the use of one of the following fastening techniques:
Welding the support element to the inner surface;
Gluing the support element to the inner surface;
Covering the support element with a textile surface and fastening, in particular sewing, gluing or joining, the textile surface to the inner surface along an edge of the support element;
Covering the support element with a shrink film and welding the textile surface to the inner surface by applying heat, pressure or laser radiation;
Sewing the support element directly to the inner surface;
Covering the support element or partially covering the edge of the support element with a compound that hardens or glues due to heat or any other influence.

For the embodiments comprising one padding element or a plurality of padding elements, the same fastening techniques can be used as described in the method described above for fastening the support element. For example, the support element, or optionally also a padding, can first be partially enclosed or folded over by a textile surface and then, together with the textile surface, be joined to the stocking cover.

In an alternative manufacturing method for manufacturing a stocking according to the invention, the manufacturing method comprises the steps of:
providing a stocking cover made of textile, wherein the stocking cover has a foot part with an inner surface,
providing a casting tool with a negative mold for accommodating a liquid precursor of the material for a support element,
stretching the stocking cover with the inside of the foot part facing the negative mold,
filling a liquid precursor of the material for a support element into the negative mold, wherein the liquid precursor comes into contact with the inner surface, and
curing of the liquid precursor to form a solid support element.

In this alternative manufacturing method, the support element is not provided before the fastening, but is only created by curing the liquid precursor material, e.g. by polymerization and/or vulcanization. In the simplest variant, this creates the connection to the inner surface of the foot part. The shape in which the stocking can be worn on the foot can then be achieved by turning the stocking cover inside out.

In variants of this manufacturing method, the mold can, for example, be positioned in such a way that the specific shape of the mold, which later faces in the direction of the foot, faces downwards in the direction of gravity. In this form, the liquid starting material will reshape the negative mold particularly precisely.

In other variants of the manufacturing method, the casting mold can be positioned in such a way that the negative mold is positioned above the inner surface when viewed in the direction of gravity. In this variant, there is particularly good contact between the textile inner surface and the liquid precursor, so that the support element adheres well to the point on the inner surface where the support element is created by curing.

In a further variant, a textile layer can be inserted into the negative mold before the precursor material is filled in. In this variant, the support element is created between the textile layer of the inner surface and the additional textile layer in a kind of pocket. This variant of the manufacturing method produces a stocking in which the foot does not come into direct contact with the support element, which users sometimes find more comfortable.

In a further variant, an adhesive film can be placed on the side of the negative mold on which the attachment to the inner surface of the foot part takes place before a precursor material is filled in. Depending on the material of the support element, the adhesive film can be used to achieve stronger adhesion to the inner surface than can be achieved by mere contact during curing. In a further step, the adhesive film is bonded to the inside of the stocking, thus fixing the support element in place.

In the event that the support element is made of a silicone-based material, silicone threads can be woven into the textile stocking cover in a further variant, particularly at the points where the support element is to be connected to the inner surface. This variant creates a particularly stable connection between the inner surface and, for example, a support element made of silicone rubber. More generally and not specifically related to silicone, textile threads coated with adhesive can be incorporated into the textile stocking cover so that the stocking cover and support element can subsequently be bonded with heat or another suitable post-treatment.

The invention further relates to a kit according to claim 19, namely to a kit for manufacturing a stocking according to the invention.

The kit according to the invention provides the essential components of the stocking, namely a textile stocking cover and a support element. Optionally, the kit may also comprise fastening means, for example a suitable adhesive for fastening the support element to the stocking.

In summary, based on the present invention, a high-quality stocking or sock can be provided which can be used to treat splayfoot and the resulting pathologies such as for example hallux valgus, hammer and claw tenosynovitis, metatarsalgia, mortoneuroma and bursitis. In addition to the possibility of treating acute splayfoot and hallux valgus, the stocking can also be used preventively. Wearing the stocking regularly can prevent the development of a deformity or reduce the progression of a slight deformity. They can be used in everyday life, sport and at work. The stocking is also easy to use and can be designed in such a way that it is not bulky and can be used as an inconspicuous therapeutic aid.

The stocking according to the invention can be designed so that it can be worn in all shoes. It can be fashionable and have an elegant and simple design. When a stocking designed in this way is worn, it conveys a lifestyle and is pleasant to walk in. The effect is immediately noticeable. The stocking according to the invention can be designed for a long service life. Machine washability can be achieved with the elements of the stocking according to the invention.

In contrast to the medical stocking from the prior art according to EP 3429514 B1, the stocking does not require a separate chamber for the big toe or other toes in order to be effective. The stocking according to the invention does not require a lateral or medial tension band, and a tension band around the heel, which exerts pressure on the Achilles tendon, can be avoided.

Exemplary embodiments of the present invention are explained in more detail below with reference to figures, wherein:

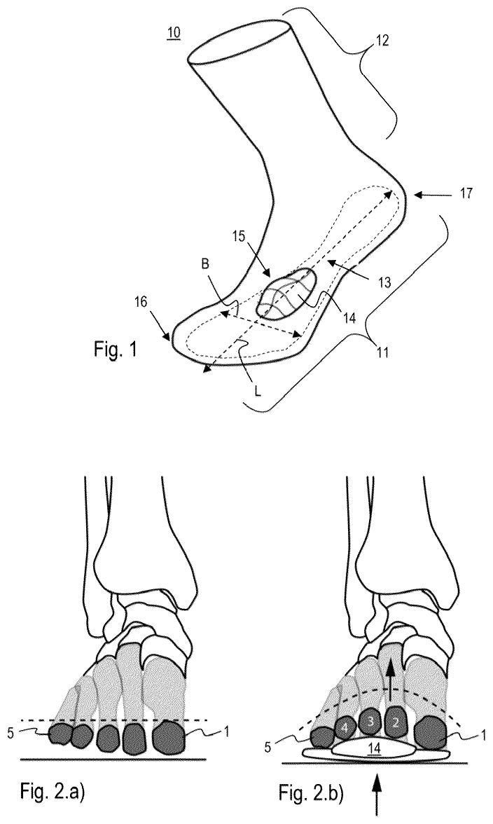

FIG. 1 shows a schematic, perspective view of a stocking according to the invention;

FIG. 2 shows cross-sections through the metatarsals to illustrate the effect of the support element, in the partial FIG. 2.a) for comparison without the support element and in 2.b) with the support element;

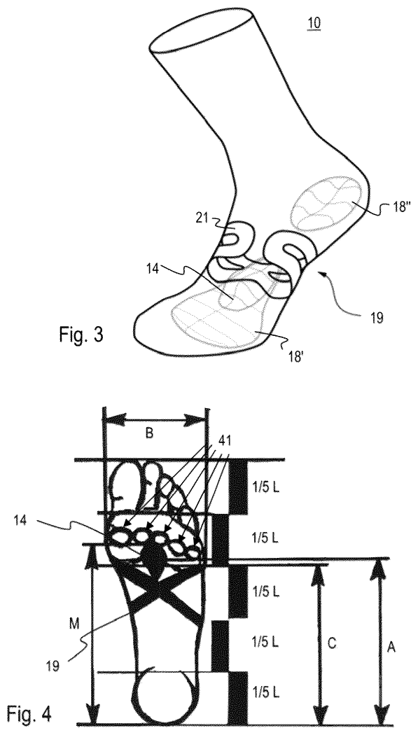

FIG. 3 shows a schematic perspective view of an embodiment of the stocking;

FIG. 4 shows a plan view of dimensions for determining the position of the support element and tensile element in relation to the foot;

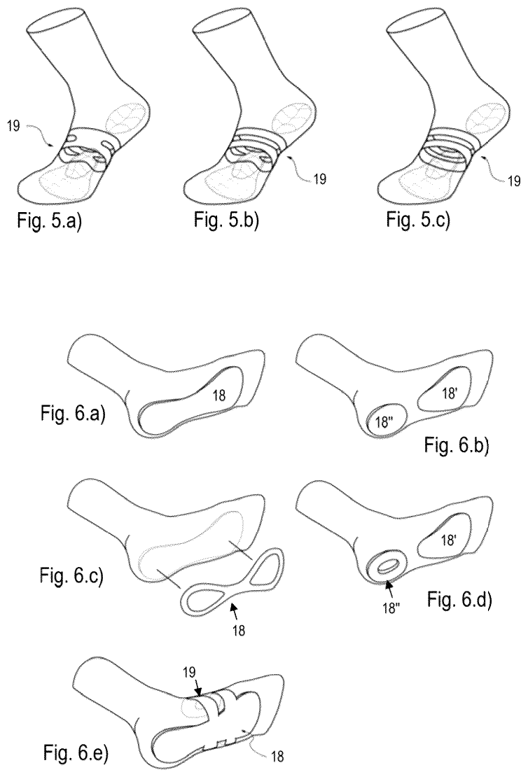

FIG. 5 shows a schematic, perspective view of variants of the tensile element;

FIG. 6 shows a schematic, perspective view of variants of position and shape paddings;

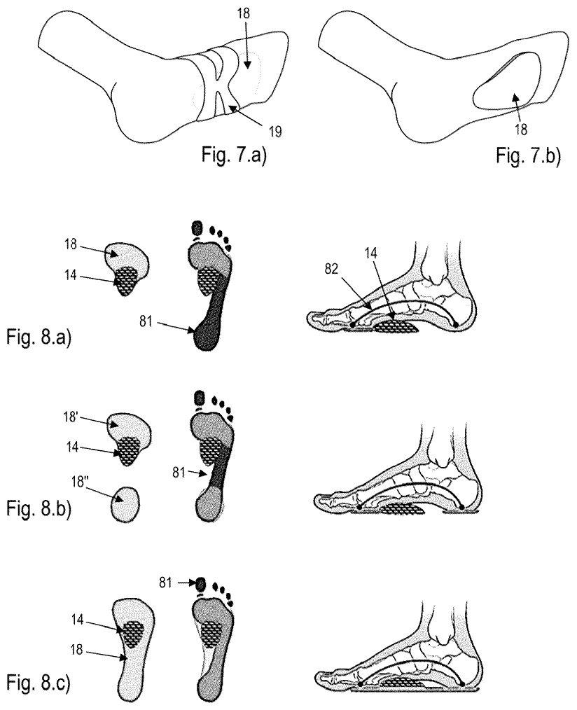

FIG. 7 shows a perspective view with the view on the sole of the foot for two examples;

FIG. 8 shows in partial FIGS. 8.a) to 8.c) variants of arrangements of support element and paddings, each in a horizontal plane and in a longitudinal section through the foot;

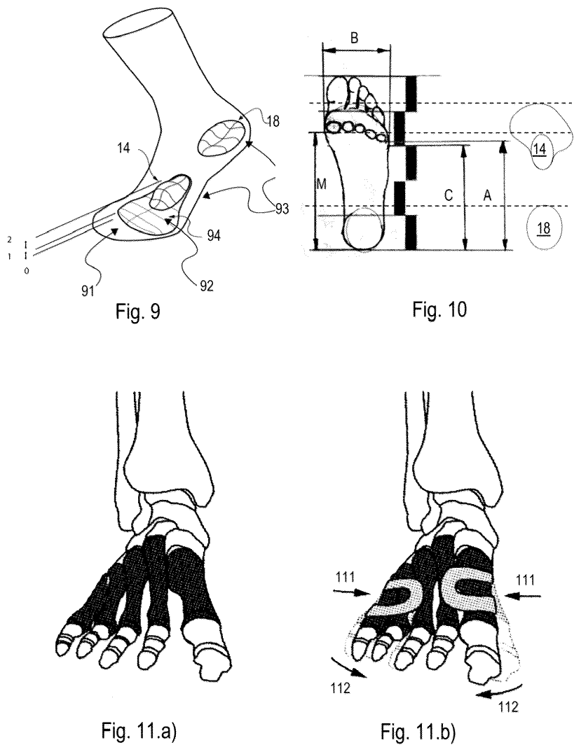

FIG. 9 shows a transparent perspective view of an embodiment of the stocking with additional height information;

FIG. 10 shows the dimensioning of a variant of an arrangement of pad and support element in relation to the anatomical features of a foot seen from a horizontal plane;

FIG. 11 shows in partial FIGS. 11.a) and 11.b) a perspective view of the effect of the tensile element on the arrangement of the bones, in particular the metatarsals, in the foot;

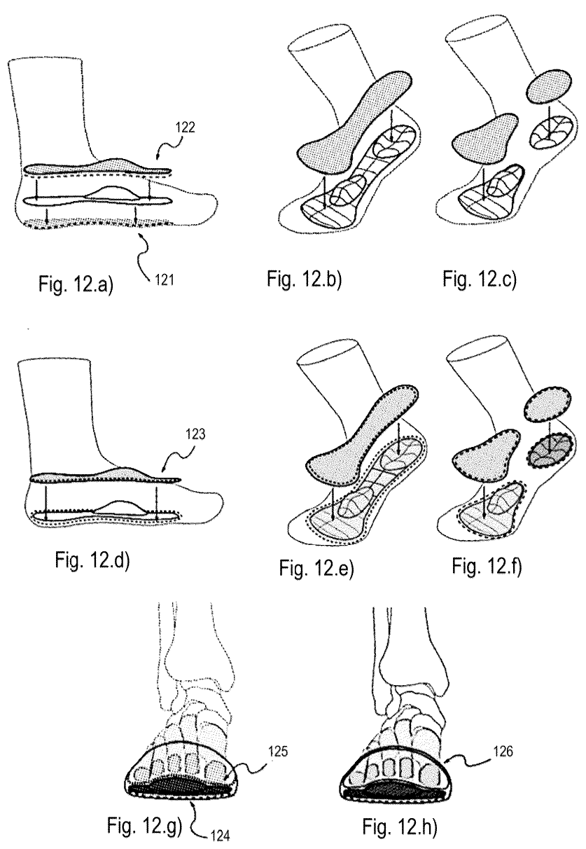

FIG. 12 shows schematic representations of method steps of a manufacturing method in side views (FIG. 12.a) and d)), in perspective view (FIG. 12.b), c), e) and f)) and in a cross-section (FIG. 12.g) and 12.h));

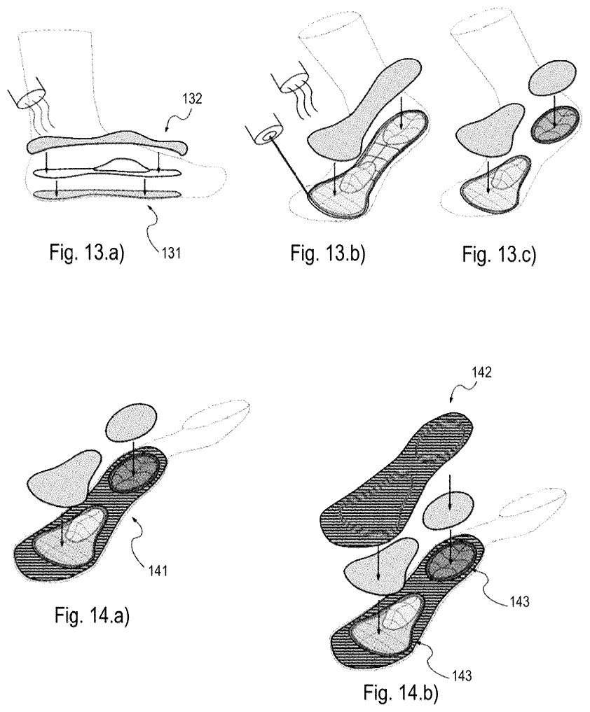

FIG. 13 shows schematic representations of alternative method steps of a manufacturing method, in side view (FIG. 13.a)) and in perspective view (FIG. 13.b) and c));

FIG. 14 shows in partial figures FIG. 14.a) and FIG. 14.b) a schematic representation of alternative method steps of a manufacturing method in perspective view;

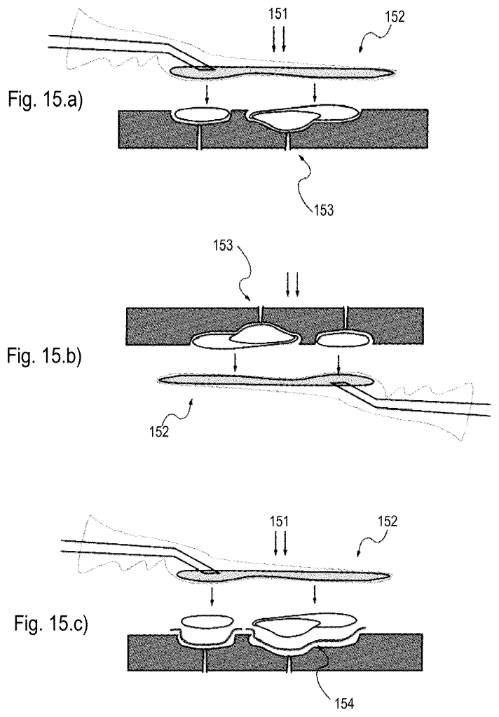

FIG. 15 shows in partial FIGS. 15.a) to 15.c) variants of manufacturing methods using a mold in a longitudinal sectional view;

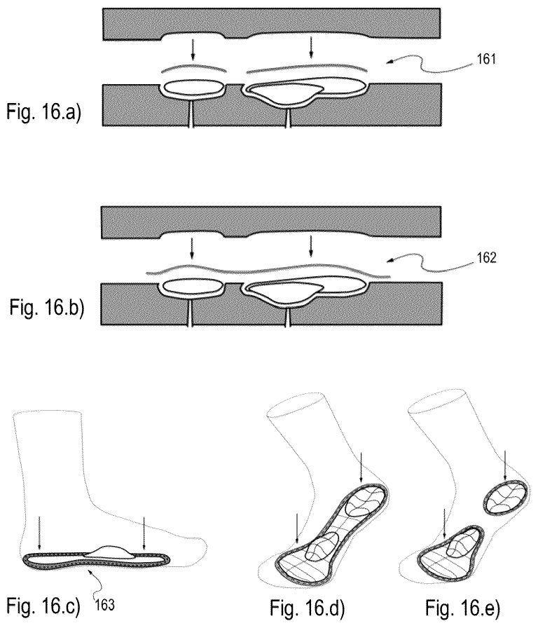

FIG. 16 shows in partial FIGS. 16.a) and 16.b) further variants of a manufacturing method using a mold in a longitudinal sectional view, in partial FIG. 16.c) in a side view and in partial FIGS. 16.d) and 16.e) in a perspective view;

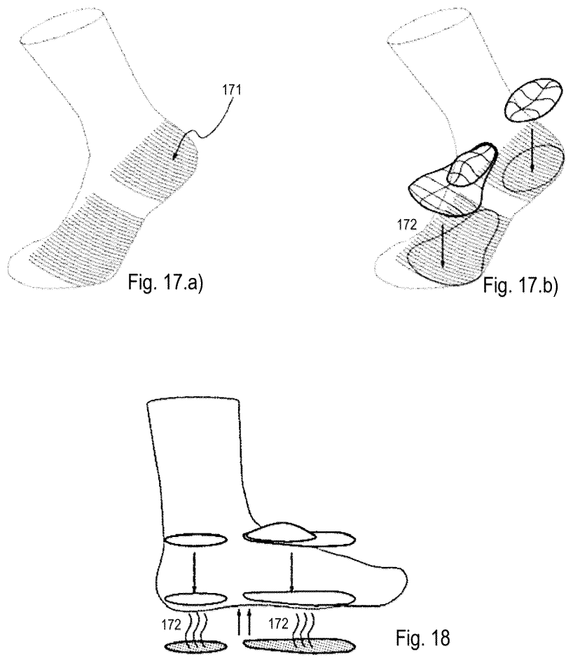

FIG. 17 shows in partial FIGS. 17.a) and 17.b) further variants of a manufacturing method in perspective view;

FIG. 18 shows a schematic representation of a further variant of a manufacturing method in side view;

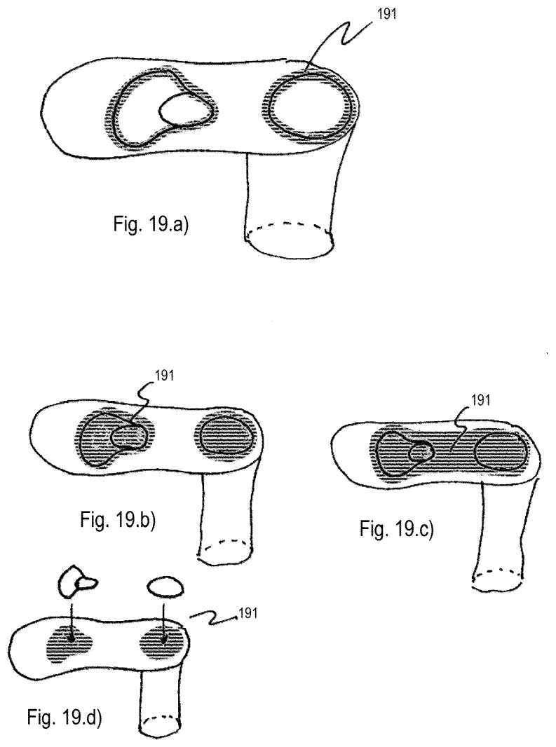

FIG. 19 shows in partial FIGS. 19.a) to 19.d) perspective views of a step in variants of the manufacturing method;

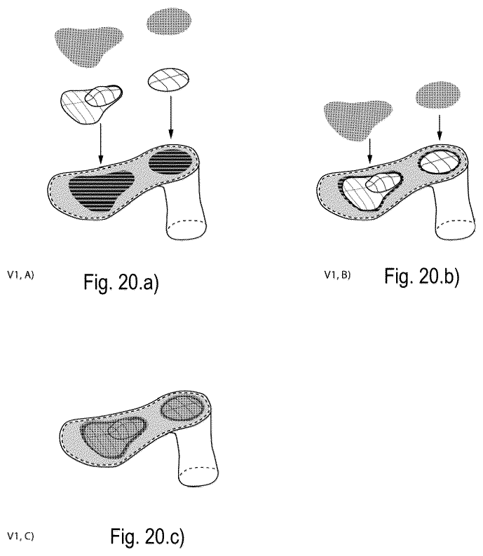

FIG. 20 shows in partial FIGS. 20.a) to 20.c) a perspective view of intermediate states in a variant of a manufacturing method;

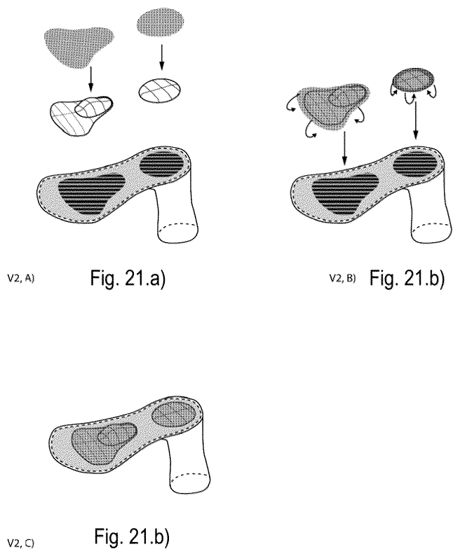

FIG. 21 shows in partial FIGS. 21.a) to 21.c) a perspective view of intermediate states in a second variant of a manufacturing method;

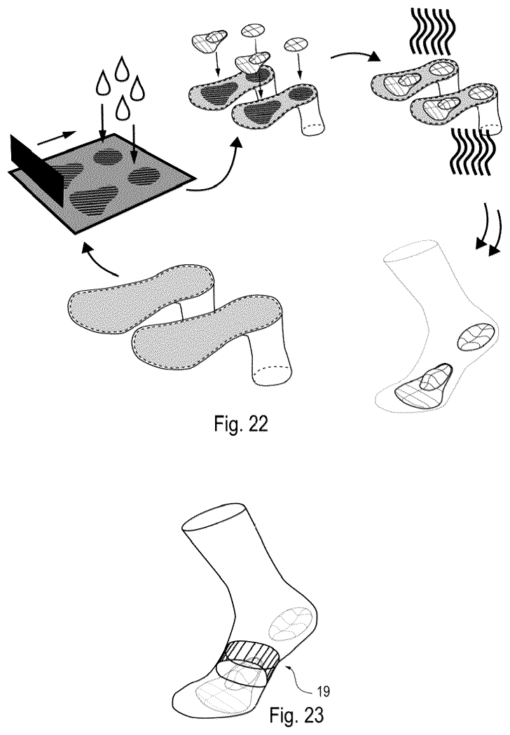

FIG. 22 shows a flow chart of a further variant of a manufacturing method;

FIG. 23 shows an embodiment of a stocking.

FIG. 1 shows a stocking 10 according to the invention, wherein the stocking is shown transparent to illustrate the position of a support element 14 on the sole area 13 of the foot part 11. The illustrated stocking has an optional leg part 12. For orientation in connection with embodiments, a first end 16 and a second end 17 of the sole area are shown here. When the stocking is worn on a foot, the toes come to lie at the first end 16 and the heel at the second end 17. The support element 14 has a curvature 15 in the direction of the inside of the stocking, wherein a possible geometry of the curvature is made visible by lines on the support element. These lines are for illustrative purposes only. The shape of the sole area 13 is shown by a dashed line. The length L and width B of the sole area are each indicated by a dashed double arrow. The shape of the stocking is shown here as if the stocking were worn on a left foot, which is not shown here.

FIG. 2.a) shows the position of the metatarsals in the event of malalignment. FIG. 2.b) shows the effect of a support element 14, which is positioned under the metatarsals 2, 3, 4. A natural arch shape of the bone arrangement is created by the force acting locally upwards on the metatarsals, as indicated by the arrows. For a better overview, the stocking to which the support element 14 is attached according to the invention is not shown here. The viewing direction is from the front onto a right foot. Other bones in the area behind the metatarsals and in the area of the ankle joint are shown for orientation.

FIG. 3 shows an embodiment of the stocking which combines various features of the embodiments discussed above. A multi-part padding 18 and a tensile element 19 interact with the support element 14. The padding comprises a forefoot pad 18′, which comes to lie in front of the support element. The padding further comprises a heel pad 18″. The tensile element has a band-shaped part 21 in the shape of the digit eight. The band-shaped part converges in the form of a crossing at a point in the sole area. Elastic fibers not shown in the stocking, which may be knitted in between the loops of digit 8, for example, close the tensile element 19 to form a ring that runs around the midfoot when the stocking is worn on the foot. This tensile element creates a force effect on the metatarsals, which further supports lifting by the support element.

In FIG. 4, dimensions are shown in plan view, which can be used to determine the position of the support element 14, here in the form of a teardrop-shaped pad, and the tensile element, here in the form of a cross-shaped converging band, in relation to the foot. The width B is transverse to the length L, which is divided into five equal sections of length ⅕ L for orientation. A dimension M, which is measured from the heel in the longitudinal direction, defines the foremost permissible position for embodiments in which the support element is to be positioned behind the metatarsophalangeal joints of the toes. Dimension A defines the center of the support element 14. Dimension C defines the front line of the position of the tensile element. Dimension A and dimension C are also measured from the heel in the longitudinal direction of the foot part. An adjustment to different sized feet can be made using the shoe size, for example, as follows, wherein the shoe sizes are grouped together.

| Shoe size | Dim. A | Dim. C | Dim. M |
|---|---|---|---|
| 36/37/38/39 | 15 cm | 13 cm | 17.0 cm |
| 40/41/42 | 16.4 cm | 14.4 cm | 18.4 cm |
| 43/44/45 | 17.6 cm | 15.6 cm | 19.6 cm |
| 46/47/48 | 18.8 cm | 16.8 cm | 20.8 cm |

In the embodiment shown in FIG. 4, the support element 14 comes to lie under the group of metatarsals comprising metatarsals no. 2 and no. 3. This is a rather small support element, the center of which is defined here by the dimension A. For a differently shaped or larger support element, for example a support element with a larger extension in the longitudinal direction, the dimension A can be adjusted accordingly. In particular, it can be taken into account that the support element should not extend below or even in front of the metatarsophalangeal joints when the stocking is worn on the foot. In a variant not shown, the tensile element can, for example, be arranged so that the tensile element on the side of the metatarsal 1 (on the side of the big toe) is positioned slightly further forward than on the side of the metatarsal 5 (on the side of the little toe). Such a variant can be described by two different dimensions in the order of magnitude of dimension C. The position of the five metatarsophalangeal joints 41 is shown. The support element 14 is therefore located behind the metatarsophalangeal joints.

FIG. 5 shows variants of the tensile element 19 in three partial figures FIG. 5.a) to FIG. 5.c). In the three variants shown, the tensile element completely surrounds the midfoot. In the variant shown in FIG. 5.a), the straps of the tensile element converge in a cross shape. A larger surface is formed on the upper side of the instep, which distributes the pressure evenly. In the variant according to FIG. 5.b), the loop runs in two parallel bands at the top, which meet crosswise on the underside of the foot. In the variant according to FIG. 5.c), the foot is compressed by means of two loops continuously extending in parallel to each other.

FIG. 6 shows in five partial figures FIGS. 6.a) to 6.e) shapes and positions of paddings 18. For reasons of better representation, other elements of the stocking according to the invention are shown here in a partly non-visible manner. The padding 18 according to FIG. 6.a) is formed in one piece and covers a large part of the sole area. FIG. 6.b) and FIG. 6.d) show a two-part padding with forefoot pad 18′ and heel pad 18″, wherein in the case of FIG. 6.d) the heel pad is ring-shaped or torus-shaped. FIG. 6.c) shows a more complex padding with hole-shaped recesses. FIG. 6.e) shows a variant in which the padding 18 is directly connected to a tensile element 19. In variants not shown for the embodiments in FIG. 6.a), 6.b), 6.d) and 6.e), the padding can extend all the way to the front, i.e. up to under the toes.

FIG. 7.a) shows a view of the sole of the foot for an example in which the straps of the tensile element 19 meet in a cross shape on the underside of the foot. An example of padding 18 is positioned here in such a way that the crossing point of the straps of the tensile element comes to lie under the padding. The padding shown here has a thickened area, not shown, with a curvature towards the inside of the stocking, which forms the support element. This is analogous to that shown in FIGS. 3 and 5.a) to 5.c). The variant of the stocking shown here does not have any heel pad. In a variant not shown, the padding 18 can extend all the way to the front, i.e. up to under the toes. FIG. 7.b) shows an embodiment with padding 18 as in FIG. 7.a), but without the tensile element.

FIG. 8.a) shows a first variant of a combination of support element 14 and padding 18. On the far left is the shape and arrangement in a plan view, with the padding 18 shown in a gray grid and the support element hatched, as in the following partial figures. In the center is the same plan view, here additionally with the tread surface 81, on which the foot treads directly, i.e. without the supporting effect of padding or support element. In this case, this tread surface comprises the toes, the heel and an area on an outer edge of the foot. The same first variant is shown on the right in a longitudinal section through the foot. The longitudinal arch 82 of the foot is schematically represented by an arc with end points in the area of the heel and behind the toe joint. The effect of the support element is to raise the middle area, in this case the first third from the left, of the longitudinal arch. The support element therefore supports the metatarsals. The padding is positioned in such a way that it cushions the lower surface of the foot at one of the points where the impact force is particularly high and where natural fatty tissue already cushions the foot.

FIG. 8.b) shows a second variant in analogous representations to the first variant. In contrast, the second variant comprises an additional padding 18″ in the heel area, i.e. a total of two paddings 18′ and 18″. The padding in the heel area is also located at a point that is already naturally padded by fatty tissue.

FIG. 8.c) shows a third variant in which the areas padded in the variant shown in FIG. 8.b) are still padded, but the padding is joined to form a continuous, single-piece surface.

FIG. 9 shows an embodiment of the stocking with a padding 18 in the heel area and a support element 14 with two height areas. Height levels “0”, “1” and “2” are indicated schematically at the front left. The hump-like elevation in the rear area of the support element reaches its full height, which in the illustrated embodiment corresponds to approximately three times the height of the extended plateau 92 of height position “1” in the front area of the support element. This means that the highest point of the support element protrudes beyond the plateau of height level “1” by twice the height of height level “1”. Specific dimensions of the support element can, for example, be selected so that the height position “1” corresponds to a height of 2-3 mm and the height position “2” corresponds to a height—measured from the very bottom—of 6-9 mm. In this embodiment, the support element is made of the same material throughout. Advantageously, this may be a silicone rubber, e.g. a silicone rubber of Shore hardness in the range 4 to 8, in particular a Shore hardness of 5. In the version shown, the support element 14, which could be described as a splay foot pad due to its mode of action, and also the padding 18 at the rear, which forms a heel pad, both lie on the inside of a textile which forms the cover of the stocking. The geometry of the front edge and the position of the support element are modeled on the anatomy of the foot in such a way that the toes do not come to rest on the support element, but instead come into contact with the textile of the stocking in the toe area 91 on the underside.

FIG. 10 shows a plan view of the positions and dimensions of a support element 14 with two height areas and a padding 18 in the heel area for an embodiment according to FIG. 9 and in relation to the dimensions of a foot, analogous to the dimensions M, B, C, A already shown in FIG. 4 in connection with another embodiment. On the right-hand side, the contours of the support element 14 and the padding 18 are shown and set in relation to the corresponding position on the foot via dashed lines. The height area 2 of the support element is located behind the toe joints and therefore remains behind the maximum distance measured from the heel, and being marked M. The front edge of the height area is designed so that the toes protrude beyond this front edge. The shape of the support element can have contours in the front area which extend in such a way that the angle from the second toe to the big toe slopes slightly backwards in relation to a longitudinal axis of the foot and the angle from the middle toes to the little toe slopes backwards. This creates a shape in which the toes are always exposed, i.e. all toes protrude beyond the support element. The front edge of the support element thus forms a kind of arrow or plow shape. This shape of the front edge can be combined in particular with a support element that has a teardrop shape over its entire outline, with the teardrop shape tapering towards the rear, i.e. towards the heel. As in FIG. 4, the position of the metatarsophalangeal joints is shown here.

FIG. 11.a) shows the position of the foot bones seen from the top front without the effect of the tensile element. The five metatarsals (metatarsal bones) are shown with a dark grid. FIG. 11.b) shows the position of the bones under the influence of a tensile element, here in a variant that is not continuously connected over the instep of the foot. The direction of action 111 of the tensile element leads to the metatarsals moving closer together and to a reduction 112 in the distance between the metatarsophalangeal joints.

FIG. 12.a) schematically shows the steps of an attachment 121 of a padding to an inner surface in the foot part by welding or gluing and a fastening 122 of a textile layer to the padding, also by welding or gluing. The cover of the stocking is shown in its final form for orientation, but may be turned inside out during the actual method steps and be present in a somewhat deformed form. FIG. 12.b) shows an embodiment with a single-piece pad and support element, FIG. 12.c) a variant with a separate heel pad and support element, both of which are covered separately with a textile. FIG. 12.d) shows a further version with sewing 123 of the textile layer over the pad. Analogous to the variants in FIGS. 12.b) and c), FIGS. 12.e) and f) show the corresponding variant with sewing along the edge of the respective areas, wherein the seam is shown by a dashed line. FIG. 12.g) shows a cross-section in which a second textile layer 15 is woven into the stocking, which is not directly visible in the drawing, and forms a chamber for the toes or the foot. The second textile layer covers the paddings and the support element like a pocket, with the foot then standing on the pocket. FIG. 12.h) shows a cross-section, wherein in this variant two complete chambers 126 or layers 126 in the stocking form a receptacle for the toes, on the one hand, and a receptacle for the support element, on the other hand.

FIGS. 13.a) to 13.c) show analogous variants to FIG. 12.a) to 12.c), wherein the fastening method used is welding with hot air (hot air dryer), pressure or laser. For this purpose, a textile and/or a shrink film is attached over the padding or the support element.

FIGS. 14.a) and 14.b) show further variants for fastening the padding and/or support element with the aid of a device on which the stocking cover is stretched with the inside facing outwards (141). For example, a plate 142 with heating elements can be used in the form of the support element or the padding in order to press the support element or the padding together with a textile or film placed over it against the stocking cover under the effect of heat.

FIG. 15 shows variants of the manufacturing method in which the support element and/or padding are produced in a mold by casting a liquid precursor of the material for the support element or the padding and then curing, in particular polymerizing and/or vulcanizing, to form the solid support element or the solid padding. The mold can be closed by applying counterpressure from the side of the stocking, and the liquid precursor material can be filled into the negative mold through the indicated channels. In the sandwich principle, the stocking can be clamped with a casting mold counterpart. Before the casting process, a textile film 154 can optionally be inserted into the mold. Both the textile material of the stocking cover and the textile film 154 bond with the support element or the padding during the curing process. Beforehand, for example, the stocking cover is inverted, i.e. stretched out on a holding device with the inside facing outwards, as schematically indicated in all three partial FIGS. 15.a) to 15.c).

FIG. 16.a) and FIG. 16.b) show further variants with casting molds, wherein in the case of FIG. 16.a) adhesive films are inserted into the respective areas of the padding and support element before the casting process in order to produce padding and support elements that can be glued in. In the case of FIG. 16.b), a textile is inserted into the mold before the casting process, which extends over the padding and support element. FIG. 16.c) to 16.d) shows the sewing steps 163 following the production by casting. The sewing can be combined with both variants shown above.

FIG. 17.a) shows a variant of the method which can be combined with previously shown variants and is particularly useful if the padding and/or support element are made of a silicone-based material. In preparation, a thread 171 with silicone adhesive or a silicone thread 171 is knitted in during the knitting process of the stocking cover. FIG. 17.b) shows the subsequent step of fastening, in this case both a support element and a heel pad made of silicone material, wherein a connection between the stocking cover and the pad or support element is produced by heating or melting silicone adhesive or silicone thread. This basic idea can also be implemented with an adhesive for materials other than silicone.

FIG. 18 illustrates the process of heating the silicone adhesive or silicone thread from the method according to FIG. 17.b) using heating devices whose geometry has been adapted.

FIG. 19 shows variants for applying a bonding compound, a coating material, an adhesive, in particular an adhesive for attaching support elements and paddings made of a polymer, in particular a silicone rubber. In the following, silicone parts are referred to in connection with the support element and the padding, even if it may be a different material. The surface 191 with adhesive is shown with horizontal hatching. In the variant according to FIG. 19.a), only the edge parts of the padding or support elements to be attached are coated with adhesive. In the variant according to FIG. 19.b), the silicone parts are completely covered with adhesive. In the variant according to FIG. 19.c), the entire sole surface and the silicone parts are coated. This coating is cured by the effect of heat or by other methods and can then serve as a bonding and cushioning material.

In the variant according to FIG. 19.d), the adhesive is first applied to the stocking cover and only then are the silicone parts applied to the adhesive layer and the adhesive allowed to cure, wherein the parts bond firmly to the textile stocking cover. As an alternative to the process shown here, the adhesive can first be applied to the silicone parts and in a further step the silicone parts provided with an adhesive layer can be contacted with the stocking cover.

FIG. 20 shows intermediate states in partial FIGS. 20.a) to 20.c), as they occur in a first variant (V1) of the connection of the support element and pad with the stocking cover and textile.

FIG. 20.a) shows the stocking cover stretched over a holding device that stabilizes the sole area. An adhesive, for example a silicone adhesive, has been applied to the sole using a coating device. The support element and pad are provided to be placed at the points coated with adhesive. Textile pieces are provided to be placed over the support element and padding.

FIG. 20.b) shows the situation after the support element and the pad have been placed on the adhesive. Adhesive surfaces protrude beyond the edge of the padding and support element, e.g. to a width of 1-5 mm. The textile pieces are now placed over the support element and padding so that they are bonded all around with the adhesive at the edge.

FIG. 21 shows intermediate states in partial FIGS. 21.a) to 21.c), as they occur in a second variant (V2) of the connection of support element and pad with stocking cover and textile.

The process in FIGS. 21.a) to 21.c) is similar to the process in FIGS. 20.a) to 20.c). In this second variant, however, the textile pieces are folded or folded over around the support element or padding so that these parts are already partially enclosed by the textile piece before they are placed and attached to the adhesive. The support element and padding are then applied to the adhesive surface and glued together with the textile piece. In this variant, the shape of the adhesive surface is adapted exactly to the contour of the support element and padding, i.e. there is no edge of the adhesive surface that protrudes beyond the parts to be attached.

FIG. 22 shows a flow chart of a further variant of a manufacturing method. The manufacturing method takes place in the steps of:
inverting the stocking, i.e. with the surface which is on the inside when the stocking is used facing outwards, and stretching it onto a holding device,
then in the coating process with adhesive or a connecting material which is liquid, for example with the aid of a template or by screen printing,
subsequently attaching the padding and support element, this step can be supported by a press, for example,
then in the curing process, for example curing by the action of heat, symbolized here by wavy lines, and
ejecting the stocking.

Subsequently, after ejection, the textile could be fixed or as a step before curing with heat, for example according to V1, as in FIG. 20, or alternatively according to V2, as in FIG. 21.

Such a process can be carried out on a so-called rotary transfer machine, for example. Automation of the process, e.g. with the help of a conveyor belt, is easy to implement.

FIG. 23 shows a further embodiment of the stocking. In this embodiment, the tensile element 19 is designed in the form of a tubular section which surrounds the foot in the region of the metatarsals. This is one of the embodiments in which the tensile element is topologically doubly contiguous, i.e. in particular no crossing point is formed on the tensile element. The relative position in relation to a support element and a padding in the heel area is recognizable in this embodiment, which is drawn as transparent for the illustration. The tensile effect of the tensile element therefore also occurs in the longitudinal axis of the foot where the support element lifts the metatarsals. The shape of the support element in the forefoot area indicated here has a cushioning effect and prevents pressure points, as does the additional padding in the heel area.

### LIST OF REFERENCE SIGNS

1 First metatarsal, behind big toe
2, 3, 4 Metatarsals 2-4
5 Fifth metatarsal
10 Stocking
11 Foot part
12 Leg part
13 Sole area
14 Support element
15 Curvature (of the support element)
16 First end (of the sole area, toes)
17 Second end (of the sole area, heel)
18 Padding
18′ Forefoot pad
18″ Heel pad
19 Tensile element
20 Band-shaped sections (of the tensile element)
21 Part in the shape of the digit eight
41 Metatarsophalangeal joints
81 Surface on which the foot treads directly
82 Longitudinal arch of the foot
91 Toe area
92 Plateau (with anatomical shape)
93 Points of contact of the foot
94 Acting as a cushion for the forefoot
111 Direction of tension around the midfoot
112 Reduction of the distance between the metatarsophalangeal joints
121 Fastening of pad to inner surface in the foot part (welded or glued)
122 Fastening of textile layer to pad (welded or glued)
123 Sewing of textile layer over pad
124 Fastening at the bottom between pad and stocking cover
125 Second textile layer forming a pocket, woven into stocking
126 Two complete chambers (layers) in the stocking
131 Padding welded/connected to the stocking using film
132 Textile/shrink film attached over padding (welded using hot air, pressure or laser)
141 Stocking clamped in reverse using a device
142 Plate with heating elements in the form of the support element or padding is pressed downwards
143 Textile film over support element or over padding welded to the edges with the stocking by applying heat
151 Pressure from above (i.e. in the direction of gravity)
152 Stocking inverted (inside-out), clamped on the holding device
153 Padding or support element cures in the casting tool and is thereby bonded directly to the stocking textile
154 Textile film inserted into the mold before the casting process
161 Adhesive film inserted into the mold before the casting process
162 Textile inserted into the mold before the casting process
163 Sewing of pad and textile
171 During the knitting process for production, a thread with adhesive, in particular silicone adhesive, or a silicone thread is knitted in as an alternative
172 Melting of adhesive, in particular silicone adhesive, or silicone thread, so that a connection is created between the stocking cover and the padding or support element.
191 Surface with adhesive and/or coating material
L Length (of the sole area)
B Width (of the sole area)
A, C, M Dimensions for determining the position of the support element and tensile element

1. Stocking with a foot part for accommodating a human foot, wherein a support element is positioned on a sole area of the foot part in such a way that the support element comes to lie at least under one of the metatarsals when the stocking is worn on the foot, wherein the support element has a curvature facing towards the inside of the stocking.

2. Stocking according to claim 1, wherein the support element is designed and positioned such that the second to fourth metatarsals are raised relative to the first and fifth metatarsals when a wearer of the stocking treads the ground.

3. Stocking (10) according to claim 1, wherein the support element (14) is designed and positioned in such a way that the support element (14) comes to lie behind the metatarsophalangeal joints when the stocking is worn on the foot.

4. Stocking according to claim 1, wherein the support element comprises a first area with a first height and a second area with a second height arranged in an island shape in the first area or at an edge of the first area, wherein the second height exceeds the first height by a factor of two.

5. Stocking according to claim 1, wherein the support element consists of a material with a Shore hardness in the range 0 to 50, in particular with a Shore hardness in the range 4 to 8.

6. Stocking according to claim 1, wherein a length of the sole area is defined from a first end for accommodating the toes to a second end for accommodating the heel, wherein a width of the sole area is defined perpendicular to the length and parallel to the sole area, and wherein the support element is centered with respect to the width and aligned with respect to the length to a position at a distance of ⅖ of the length from the first end and ⅗ of the length from the second end.

7. Stocking according to claim 1, wherein the support element has a teardrop shape when viewed perpendicular to the sole area, wherein the teardrop shape is oriented such that a tapering area of the teardrop shape faces towards the heel when the stocking is worn on the foot.

8. Stocking according to claim 1, wherein the stocking has a one-piece or multi-piece padding (18) on the sole area (13).

9. Stocking according to claim 8, wherein at least a part of the padding is formed as forefoot padding, wherein the forefoot padding is arranged at the sole area in such a way that it comes to lie under the metatarsophalangeal joints when the stocking is worn on the foot.

10. Stocking according to claim 1, wherein the foot part of the stocking comprises a tensile element which is arranged on the foot part or incorporated into the foot part such that the tensile element extends around the midfoot and exerts a contracting force on the midfoot which is greater than a contracting force of the remaining foot part when the stocking is worn on the foot.

11. Stocking according to claim 10, wherein the tensile element comprises band-shaped sections which converge in a cross shape in the sole area or which run parallel in the sole area or which converge in a cross shape in the region of the foot instep.

12. Stocking according to claim 10, wherein the tensile element comprises a band-shaped part which, in unwound form, has the shape of the digit eight.

13. Stocking according to claim 10, wherein the tensile element is topologically doubly connected.

14. Stocking according to claim 8, wherein the tensile element comprises a first section along a circumference extending around the foot part having less extensibility than the rest of the foot part and comprises a second elastic section, wherein the first and the second section together form a closed ring.

15. Stocking according to claim 1, wherein the stocking comprises a clasp element which is designed to at least partially embrace the metatarsals and exert a force acting laterally on the foot when the stocking is worn on the foot.

16. Stocking with a foot part for accommodating a human foot, wherein a pad is attached to a sole area of the foot part in the region of the forefoot.

17. Manufacturing method for manufacturing a stocking according to claim 1, wherein the manufacturing method comprises the steps of
providing a stocking cover made of textile, wherein the stocking cover has a foot part with an inner surface,
providing a support element, and
fixing the support element to the inner surface of the foot part of the stocking cover, wherein the fixing step comprises the application of a fastening technique from the following group:
welding the support element to the inner surface,
gluing the support element to the inner surface,
covering the support element with a textile surface and fastening, in particular sewing, gluing or joining, the textile surface to the inner surface along an edge of the support element,
covering the support element with a shrink film and welding the textile surface to the inner surface by applying heat, pressure or laser radiation,
sewing the support element directly to the inner surface
covering the support element or partially covering the edge of the support element with a compound that hardens or glues by heat or any other influence.

18. Manufacturing method for manufacturing a stocking according to claim 1, wherein the manufacturing method comprises the steps of
providing a stocking cover made of textile, wherein the stocking cover has a foot part with an inner surface,
providing a casting tool with a negative mold for accommodating a liquid precursor of the material for a support element,
stretching the stocking cover with the inside of the foot part facing the negative mold,
filling a liquid precursor of the material for a support element into the negative mold, wherein the liquid precursor comes into contact with the inner surface, and
curing of the liquid precursor to form a solid support element.

19. Kit of parts for manufacturing a stocking according to claim 1, wherein the kit of parts has at least one textile stocking cover with an inner side of the stocking and a support element, wherein the support element has a curvature and is attachable to the stocking cover in such a way that the curvature faces the inside of the stocking.
